(19) United States
(12) Patent Application Publication
(10) Pub. No.: US 2001/0046478 A1
(43) Pub. Date: Nov. 29, 2001

(54) Antiinfective combinations and their use for the topical treatment of fungal infections of the toenails and fingernails

(76) Inventors: Manfred Bohn, Hofheim (DE); Karl Theodor Kraemer, Langen (DE)

Correspondence Address: Finnegan, Henderson, Farabow, Garrett & Dunner, L.L.P., 1300 I Street, N.W., Washington, DC, 20005-3315, US

(21) Appl. No.: 09/799,726

(22) Filed: Mar. 7, 2001

(30) Foreign Application Priority Data
Mar. 9, 2000 (DE) 10011081.9

(51) Int. Cl. A61K 7/04; A61K 31/495; A61K 31/44

(52) U.S. Cl. 424/61; 514/345; 514/254.07

(57) ABSTRACT

A preparation comprising a combination of a topical and a systemic antimycotic and a physiologically acceptable lacquer base is suitable for the treatment and prophylaxis of onychomycoses. Preference is given to the use of water-insoluble lacquer preparations and a combination of at least one systemic antimycotic from the group of itraconazole, terbinafine and fluconazole or salts thereof with at least one topical antimycotic from the group of ciclopirox, 6-(2,4,4-trimethylpentyl)-1-hydroxy-4-methyl-2(1H)-pyridone, amorolfine and butenafine or salts thereof.

The present application claims benefit of priority of German patent application No. 1001 1081.9, filed Mar. 9, 2000, the disclosure of which is incorporated herein by reference in its entirety.

Fungal infections of the toenails and fingernails (onychomycoses) are widespread around the world. This chronic pathological entity, which does not tend to heal by itself, is becoming increasingly important particularly in highly developed industrialized countries. Onychomycoses constitute the most common disorder of nails, comprising a proportion of up to 40%. The prevalence of onychomycoses is stated in the state of the art to be 2.8% to 8.4%. Mycoses of nails now account for about 30% of all dermatomycoses. Epidemiological studies show that 20% to 30% of patients with Tinea pedis also have onychomycosis.

Many patients feel restricted in their social contacts especially when the onychomycosis is located on the fingernails where it is clearly visible. In addition, the pathological event results in a possible restriction of tactility, motility and manual abilities. The need for treatment also derives from the fact that onychomycoses contribute, as source of infection, to the spread of the disease from the nail to the free skin. In addition, they represent a risk of infection for a continually increasing population.

Since the early 1990s, a large number of new treatment methods for the therapy of onychomycosis has been developed. These are, on the one hand, novel systemically active antimycotics; for example, itraconazole, see U.S. Pat. No. 4,267,179; (E)-N-(6,6-dimethyl-2-hepten-4-ynyl)-N-methyl-1-naphthalene-methanamine, which is also called terbinafine, and α-(2,4-difluorophenyl)-α-(1H-1,2,4-triazol-1-ylmethyl)-1H-1,2,4-triazol-1-ethanol, which is also called fluconazole, but also lacquer preparations to be used topically, which make the treatment of onychomycoses more promising.

Experience has shown that systemic therapy may occasionally lead to serious, unwanted side effects of pharmaceuticals which may, in some circumstances, be life-threatening, because the active ingredient must reach the site of infection via the blood circulation. Side effects and interactions with other medicines are unavoidable in particular in many elderly patients with multimorbidity. Systemic antimycotics additionally have other unwanted concomitant effects such as gaps in the range of pathogens which can be treated or, in some cases, unreliable absorption. Various studies have very recently been carried out with antimycotic-containing lacquer preparations in combination with systemic itraconazole or terbinafine therapy on patients with pronounced onychomycosis. The results show that the combination of a topical lacquer preparation with a systemic administration of itraconazole or terbinafine is distinctly superior to monotherapy with itraconazole and terbinafine for the therapy of severe onychomycoses. Results to date indicate that the rate of unsuccessful systemic therapy of onychomycoses can be considerably reduced by combination therapy with a topically administered antimycotic-containing lacquer preparation and an antimycotic administered systemically.

A disadvantage of combined treatment with a topically administered antimycotic and a systemically administered antimycotic in the therapy of onychomycoses is still the stress on the system with all the possible adverse effects connected therewith for the patient even with this treatment strategy. Another important disadvantage here is the small amount of systemically administered antimycotic which reaches the toenail or fingernail. In addition, it has to date been possible only with very complicated and drastic methods to apply systemically active antimycotics such as itraconazole in therapeutically effective concentrations topically to the toenail or fingernail (see WO 96/19186).

The present invention provides, in one of its many embodiments, a composition which tends to avoid the known disadvantages associated with the described topical/systemic combination therapy of onychomycoses.

An existing technical prejudice holds that systemically active antimycotics such as itraconazole penetrate into the nail in sufficient concentration on topical application only after breaking the sulfur bridges in the nail keratin and through addition of urea (WO 96/19186). It has now been found, surprisingly, that combinations of topically active antimycotics with systemically active antimycotics in a lacquer base are able after topical application to penetrate in and through the nail in therapeutically effective concentrations. This penetration may occur without breaking the sulfur bridges of the nail keratin, and does not require the presence of urea. In contrast, patients treated with a systemically administered antimycotic show only comparatively low concentrations of the antimycotic in the toenail or fingernail.

As used herein, “topical antimycotic” refers to a substance exhibiting fungistatic and/or fungicidal properties when applied topically to a patient. This term is synonymous with “topically active antimycotic” and “topical antimycotic active ingredient.”

“Systemic antimycotic” refers to a substance known to exhibit fungistatic and/or fungicidal properties when administered systemically to a patient.

One of ordinary skill in the art will recognize, where appropriate, that “systemic antimycotics” refers to antimycotics which were not usually administered topically, or if they were, they were subject to the aforementioned or other technical prejudices.

Similarly, as used herein, “topical antimycotics” refers to antimycotics which were not usually administered systemically, or if they were, they were subject to technical prejudices against their use as systemic antimycotics.

“Antimycotics,” unless otherwise apparent, refers to both “topical antimycotics” and “systemic antimycotics.”

The invention therefore relates to, among many embodiments, compositions comprising at least one topical antimycotic and at least one systemic antimycotic, in a physiologically acceptable lacquer base.

A “physiologically acceptable lacquer base” is a substance, in some embodiments a liquid, sol, aerosol, vapor, solid, powder, gel, cream, or lotion, which when applied to a surface such as a nail, gels, dries, hardens, solidifies, deposits, spreads, or otherwise forms a film, a layer of “nail polish,” or a lacquer on or with the surface. Also, this substance does not usually cause unbearable injury or irritation to an average patient, such that the patient could not withstand exposure to the substance. In some embodiments, the physiologically acceptable lacquer base is chosen to be well-tolerated by a patient, perhaps in view of the patient's idiosyncratic reaction to the composition, including but not limited to the patient's allergies and/or aesthetic sensibilities. The physiologically acceptable nail lacquer optionally comprises one or more film formers, which are described below.

The invention relates in another embodiment to a composition comprising a water-insoluble film former, at least one systemic antimycotic from the group of itraconazole, terbinafine and fluconazole and/or physiologically tolerated salts of itraconazole, terbinafine and fluconazole, and at least one topical antimycotic from the group of ciclopirox, 6-(2,4,4-trimethylpentyl)-1-hydroxy-4-methyl-2(1H)-pyridone, amorolfine and butenafine and/or physiologically tolerated salts of ciclopirox, 6-(2,4,4-trimethylpentyl)-1-hydroxy-4-methyl-2(1H)-pyridone, amorolfine and butenafine.

Other embodiments include compositions which comprise ciclopirox (which is also called 6-cyclohexyl-1-hydroxy-4-methyl-2(1H)-pyridone) and itraconazole, or butenafine hydrochloride and fluconazole, or ciclopirox and fluconazole, or amorolfine hydrochloride and terbinafine hydrochloride, as antimycotic.

These and other suitable antimycotics are synthesized by methods known in the art, and many are also commercially available.

The abovementioned topical or systemic antimycotics are employed both in free form and as physiologically tolerated salts. If organic bases are used for the salts, the bases employed include bases of low volatility, for example, low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Further bases of low volatility which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine. The salts of quaternary ammonium hydroxides such as trimethylbenzylammonium hydroxide, tetramethylammonium hydroxide or tetraethylammonium hydroxide can also be used, as can guanidine and its derivatives, in particular its alkylation products.

However, it is also possible to employ as salt formers, for example, low molecular weight alkylamines such as methylamine, ethylamine or triethylamine. Salts with inorganic cations, for example, alkali metal salts, such as sodium, potassium or ammonium salts, in particular hydrochlorides, alkaline earth metal salts such as magnesium or calcium salts, and salts with doubly charged to quadrupally charged cations, for example, zinc, aluminum or zirconium salts, are also suitable salts to be employed according to the invention.

The invention further relates to the use of a combination of a topical and a systemic antimycotic in a physiologically acceptable lacquer base for producing a composition for the prophylactic and/or therapeutic treatment of fungal infections of the toenails and/or fingernails.

The invention also relates to the use of a water-insoluble film former, at least one systemic antimycotic from the group of itraconazole, terbinafine and fluconazole and/or physiologically tolerated salts of itraconazole, terbinafine and fluconazole, and at least one topical antimycotic from the group of ciclopirox, 6-(2,4,4-trimethylpentyl)-1-hydroxy-4-methyl-2(1H)-pyridone, amorolfine and butenafine and/or physiologically tolerated salts of ciclopirox, 6-(2,4,4-trimethylpentyl )-1-hydroxy-4-methyl-2 (1H )-pyridone, amorolfine and butenafine, for producing a composition for the prophylactic and/or therapeutic treatment of fungal infections of the toenails and/or fingernails.

The amount of topical antimycotic and systemic antimycotic in the compositions according to the invention depends on the structure of each antimycotic and thus on its release from the lacquer film, its penetration characteristics in the nail, and its antifungal properties.

In further embodiments of the invention, the topical antimycotic and the systemic antimycotic are present in a composition according to the invention in an amount effective for treating onychomycosis. In one embodiment, the topical antimycotic is present in an amount ranging from 0.25 to 20 percent by total weight. Here, total weight means the weight of the composition, including all volatile and involatile ingredients necessarily or optionally present, and of the antimycotics present. In another embodiment, the topical antimycotic is present in an amount ranging from 2 to 15 percent by total weight. The amount of systemic antimycotic, in another embodiment of the invention, ranges from 0.05 to 10 percent by total weight. In yet another embodiment, the amount of systemic antimycotic present ranges from 0.1 to 5 percent by total weight.

Some of the compositions according to the invention useful as nail lacquers may further comprise one or more film formers which, after drying of the composition, form a film on the nail, the film being water soluble or water-insoluble. Thus, according to the present invention, “water-insoluble film former” means a substance, which is soluble or insoluble in water, that forms a water-insoluble film when applied alone and/or in a composition to a surface such as a nail.

Substances suitable as film formers are any that form a film when applied alone and/or in a composition to a surface such as a nail. Examples of substances suitable as film formers include cellulose nitrate, and physiologically acceptable polymers such as those useful, for example, in cosmetics. Mixtures of two or more of these substances are possible. For example, one of these substances (other than cellulose nitrate) may be mixed with cellulose nitrate. Mention may be made, for example, of polyvinyl acetate and partially hydrolyzed polyvinyl acetate, copolymers of vinyl acetate on the one hand and acrylic acid or crotonic acid or monoalkyl maleate on the other hand, ternary copolymers of vinyl acetate on the one hand and crotonic acid and vinyl neodecanoate or crotonic acid and vinyl propionate on the other hand, copolymers of methyl vinyl ether and monoalkyl maleate, such as monobutyl maleate, copolymers of fatty acid vinyl ester and acrylic acid or methacrylic acid, copolymers of N-vinylpyrrolidone, methacrylic acid and alkyl methacrylate, copolymers of acrylic acid and methacrylic acid or alkyl acrylate or alkyl methacrylate, polyvinyl acetates, polyvinyl butyrals, alkyl-substituted poly-N-vinylpyrrolidones, alkyl esters from copolymers of olefins and maleic anhydride, and products of the reaction of rosin with acrylic acid. In some embodiments, the alkyl radicals in the esters are usually short-chain and mostly have not more than four carbon atoms.

Some of the embodiments of the invention which are compositions comprise a water-insoluble film former chosen from copolymer of ethyl acrylate/methyl methacrylate/trimethylammonioethyl methacrylate chloride, copolymer of acrylic and methacrylic ester with proportions of trimethylammonioethyl methacrylate chloride, copolymer of methyl vinyl ether and monobutyl maleate, polymer of polyvinylbutyral and cellulose nitrate or copolymer of methacrylic acid and ethyl acrylate.

Compositions according to the invention optionally further comprise one or more solvents. A solvent is a substance which mixes with, dissolves, solvates, suspends, softens and/or liquefies the other ingredients present in a composition according to the invention so that such composition may be topically applied to a surface such as a nail. The one or more solvents may be physiologically acceptable. Suitable physiologically acceptable solvents include the hydrocarbons, halogenated hydrocarbons, alcohols, ethers, ketones and esters useful in cosmetics, such as acetic esters of monohydric alcohols such as ethyl and butyl acetates, optionally mixed with aromatic hydrocarbons such as toluene and/or alcohols such as ethanol or isopropanol. Alcohols such as isopropyl alcohol and ethanol are also physiologically acceptable solvents.

The optional one or more solvents may help determine the drying time, spreadability and other properties of the lacquer or lacquer film. In some embodiments, the one or more solvents consist of a mixture of low boilers (which are solvents with a boiling point of up to 100° C.) and medium boilers (which are solvents with a boiling point of up to 150° C.), and optionally with a small proportion of high boilers (which are solvents with a boiling point of up to 200° C).

The compositions according to the invention may additionally comprise additives useful in cosmetics, such as phthalate- and camphor-based plasticizers, dyes and colored pigments, pearlescent agents, sedimentation inhibitors, sulfonamide resins, silicates, fragrances, wetting agents such as sodium dioctyl sulfosuccinate, lanolin derivatives, photoprotective agents such as 2-hydroxy-4-methoxybenzophenone, substances with antibacterial activity, and substances with keratolytic and/or keratoplastic effect, such as urea, allantoin, enzymes and salicylic acid.

Compositions according to the invention are optionally colored or pigmented. Colored or pigmented compositions have the advantage, for example that the composition according to the invention can be suited to the patient's esthetic perception, and the existing changes in the nails tend not to be directly visible to other people.

The invention also relates to a process for producing compositions according to the invention. In one embodiment, a process for producing a composition according to the invention, for example, comprises mixing a physiologically acceptable lacquer base with the antimycotics, and further processing the composition if necessary. The physiologically acceptable lacquer base and antimycotics may be in dissolved form.

The antimycotics are present in a composition according to the invention in an amount effective for treating and/or preventing onychomycosis. In some embodiments, the antimycotics are present in the compositions according to the invention in an amount ranging from 2 to 80 percent by weight, wherein “weight” here refers to the weight of the involatile ingredients. In other embodiments, the antimycotics are present in an amount ranging from 10 to 60 percent by weight of involatile ingredients. In yet other embodiments, the antimycotics are present in an amount ranging from 20 to 40 percent by weight of involatile ingredients. Weight of the involatile ingredients is the weight of the lacquer base which optionally comprises film formers, pigments, plasticizers and other involatile additives, and of the antimycotics present. These weights can be thought of as the ratio of antimycotic in the composition after the composition has been deposited on a surface such as a nail, and any volatile ingredients, if present, have evaporated or left the composition.

It is possible with the compositions according to the invention to achieve a partial or thorough cure on treatment of onychomycoses—with a reduced or eliminated occurrence of systemic side effects and drug interactions. In the light of experience with therapy to date, this is an exceptionally important finding. A further potential advantage is the slightly or considerably shorter treatment time which may be possible with the compositions according to the invention. This shorter treatment time may be possible given the considerably higher concentrations of the systemic antimycotics in the nail after topical application. Thus, the invention also relates to methods for treating onychomycoses, such methods comprising administering to a patient in need of such treatment an effective amount of a composition according to the invention.

Some of the compositions according to the invention are suitable for prophylactic use against onychomycoses, in which case a sufficiently large deposit of antimycotics is achieved in the nail so that, in the event of fungal contamination, there is no outbreak of a nail infection caused by fungi. The compositions useful for prophylaxis comprise antimycotics in an amount effective for the prophylaxis. Such amount effective for the prophylaxis is optionally a lesser amount of the antimycotics than the amount employed for therapy. The topical antimycotic is employed in certain embodiments of the invention useful for prophylaxis in an amount ranging from 0.25 to 4 percent by total weight. In other embodiments, the topical antimycotic is present in an amount ranging from 1 to 4 percent by total weight. In some embodiments of the invention useful for prophylaxis, the amount of systemic antimycotic ranges from 0.05 to 3 percent by total weight. In other embodiments, the amount of the systemic antimycotic ranges from 0.1 to 1 percent by total weight.

Patients who may be selected for prophylactic therapy include those who are suffering from or who have a history of Tinea pedis, fungal infections other than onychomycoses, and known or suspected susceptibility to fungal infections or onychomycoses. Also, those suffering from onychomycoses may apply compositions according to the present invention prophylactically to healthy or uninfected nails to inhibit the spread of the onychomycoses. Elderly patients and immunocompromized patients are also candidates for prophylactic therapy according to the present invention.

The invention also relates to the use of the preparations according to the invention in cosmetics. One of ordinary skill in the art will recognize that the compositions of the present invention can be added to or formed with compositions that are primarily useful for cosmetic applications, that is, cosmetic compositions. Thus, the invention also relates to a method of employing a composition according to the invention, wherein the method comprises incorporating the composition in a cosmetic composition.

Unless otherwise indicated all numbers expressing quantities of ingredients, molecular weights, reaction conditions, and so forth used in the specification and claims are to be understood as being modified in all instances by the term “about.” Accordingly, unless indicated to the contrary, the numerical parameters set forth in the present specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

The present invention is explained in detail by the following examples, but is not confined in scope to these examples. Unless otherwise noted, the stated amounts are based on total weight.

### EXAMPLE 1

| | |
|---|---|
| Amorolfine hydrochloride | 5.0% |
| Terbinafine hydrochloride | 2.5% |
| Copolymer of acrylic and methacrylic ester with | |
| proportions of trimethylammonioethyl methacrylate | 20.0% |
| chloride (e.g. EUDRAGIT RL 100) | |
| Isopropyl myristate | 2.5% |
| Isopropyl alcohol | 70.0% |

### EXAMPLE 2

| | |
|---|---|
| Butenafine hydrochloride | 5.0% |
| Fluconazole | 5.0% |
| Copolymer of methyl vinyl ether and monobutyl maleate | 25.0% |
| 96% ethanol | 65.0% |

### EXAMPLE 3

| | |
|---|---|
| Ciclopirox | 8.0% |
| Itraconazole | 0.5% |
| Isopropyl myristate | 5.0% |
| 1,2-propylene glycol | 4.0% |
| Copolymer of methyl vinyl ether and monobutyl maleate | 7.5% |
| Ethyl acetate | 25.0% |
| Isopropyl alcohol | 50.0% |

### EXAMPLE 4

| | |
|---|---|
| Ciclopirox | 7.5% |
| Fluconazole | 5.0% |
| Copolymer of methyl vinyl ether and monobutyl maleate | 15.0% |
| Ethyl acetate | 30.0% |
| 96% ethanol | 42.5% |

### EXAMPLE 5

The amount of antimycotic in the nail after application of the compositions according to the invention was determined by HPLC measurements after extraction of distal nail material obtained on cutting the nails of volunteers.

After treatment 2× a week for 6 weeks, the values found for the composition of Example 3 were as follows:

| | |
|---|---|
| itraconazole | 800 ng/g of nail |
| ciclopirox | 140 μg/g of nail |

After oral administration of 100 mg of itraconazole each day for 3 months, the itraconazole concentration in the distal nail plate is reported to be 100 ng/g of nail. See Willemsen M., De Doncker P., Willems J., et al. Post-Treatment Itraconazole Levels in the Nail. J. Am. Acad. Dermatol. 1992; 26:731-735. It is therefore possible by administering a composition according to the invention to achieve in a short time itraconazole concentrations in the nail which are about eight times higher than through systemic treatment.

### EXAMPLE 6

After treatment 2× a week for 2 weeks, the values found for the composition of Example 4 were as follows:

| | |
|---|---|
| fluconazole | 17 μg/g of nail |
| ciclopirox | 92 μg/g of nail |

Patent claims:

1. A composition comprising
at least one topical antimycotic;
at least one systemic antimycotic; and
at least one physiologically acceptable lacquer base.

2. The composition as claimed in claim 1,
wherein the at least one physiologically acceptable lacquer base comprises at least one water-insoluble film former.

3. The composition as claimed in claim 1, wherein the at least one topical antimycotic is chosen from ciclopirox, 6-(2,4,4-trimethylpentyl)-1-hydroxy-4-methyl-2(1H)-pyridone, amorolfine, butenafine, and physiologically tolerated salts of ciclopirox, 6-(2 ,4,4-trimethylpentyl)-1-hydroxy-4-methyl-2(1H)-pyridone, amorolfine and butenafine.

4. The composition as claimed in claim 1, wherein the at least one systemic antimycotic is chosen from itraconazole, terbinafine, fluconazole, and physiologically tolerated salts of itraconazole, terbinafine and fluconazole.

5. The composition as claimed in claim 1, comprising
ciclopirox and itraconazole, or
butenafine hydrochloride and fluconazole, or
ciclopirox and fluconazole, or
amorolfine hydrochloride and terbinafine hydrochloride.

6. The composition as claimed in claim 2, wherein the at least one water-insoluble film former is chosen from cellulose nitrate, polyvinyl acetate and partially hydrolyzed polyvinyl acetate, copolymers of vinyl acetate and acrylic acid or crotonic acid or monoalkyl maleate, ternary copolymers of vinyl acetate and crotonic acid and vinyl neodecanoate or crotonic acid and vinyl propionate, copolymers of methyl vinyl ether and monoalkyl maleate, copolymers of fatty acid vinyl ester and acrylic acid or methacrylic acid, copolymers of N-vinylpyrrolidone, methacrylic acid and alkyl methacrylate, copolymers of acrylic acid and methacrylic acid or alkyl acrylate or alkyl methacrylate, polyvinyl acetates, polyvinyl butyrals, alkyl-substituted poly-N-vinylpyrrolidones, and alkyl esters from copolymers of olefins and maleic anhydride, and products of the reaction of rosin with acrylic acid.

7. The composition as claimed in claim 6, wherein within the copolymers of methyl vinyl ether and monoalkyl maleate, the monoalkyl maleate comprises monobutyl maleate.

8. The composition as claimed in claim 6, wherein the alkyl esters comprise alkyl radicals which contain not more than four carbon atoms each.

9. The composition as claimed in claim 6, wherein the at least one film former is chosen from copolymers of ethyl acrylate/methyl methacrylate/trimethylammonioethyl methacrylate chloride, copolymers of acrylic and methacrylic ester with proportions of trimethylammonioethyl methacrylate chloride, copolymers of methyl vinyl ether and monobutyl maleate, polymers of polyvinylbutyral and cellulose nitrate and copolymers of methacrylic acid and ethyl acrylate.

10. The composition as claimed in claim 1, wherein the at least one topical antimycotic is present in an amount ranging from 0.25 to 20 percent by total weight.

11. The composition as claimed in claim 10, wherein the at least one topical antimycotic is present in an amount ranging from 2 to 15 percent by total weight.

12. The composition as claimed in claim 1, wherein the at least one systemic antimycotic is present in an amount ranging from 0.05 to 10 percent by total weight.

13. The composition as claimed in claim 12, wherein the at least one systemic antimycotic is present in an amount ranging from 0.1 to 5 percent by total weight.

14. The composition as claimed in claim 1, wherein the at least one topical antimycotic and the at least one systemic antimycotic are present in an amount effective for the treatment of a fungal infection of a toenail or a fingernail.

15. The composition as claimed in claim 1, wherein the at least one topical antimycotic and the at least one systemic antimycotic are present in an amount effective for the prophylaxis of a fungal infection of a toenail or a fingernail.

16. A process for producing a composition as claimed in claim 1, which comprises mixing the at least one physiologically acceptable lacquer base with the at least one topical antimycotic and the at least one systemic antimycotic.

17. The process as claimed in claim 16, wherein the at least one physiological acceptable lacquer base, at least one topical antimycotic, and at least one systemic antimycotic are present in dissolved form.

18. The process as claimed in claim 16, wherein the at least one topical antimycotic and the at least one systemic antimycotic are present in an amount effective for the treatment of a fungal infection of a toenail or a fingernail.

19. The process as claimed in claim 16, wherein the at least one topical antimycotic and the at least one systemic antimycotic are present in an amount effective for the prophylaxis of a fungal infection of a toenail or a fingernail.

20. The process as claimed in claim 16, wherein the at least one physiologically acceptable lacquer base comprises at least one water-insoluble film former.

21. The process as claimed in claim 16, wherein the at least one systemic antimycotic is chosen from itraconazole, terbinafine and fluconazole, and physiologically tolerated salts of itraconazole, terbinafine and fluconazole.

22. The process as claimed in claim 16, wherein the at least one topical antimycotic is chosen from ciclopirox, 6-(2,4,4-trimethylpentyl)-1-hydroxy-4-methyl-2(1H)-pyridone, amorolfine, butenafine, and physiologically tolerated salts of ciclopirox, 6-(2,4,4-trimethylpentyl)-1-hydroxy4-methyl-2(1H)-pyridone, amorolfine and butenafine.

23. A method of prophylaxis of an infection of a toenail or a fingernail, wherein the method comprises topically administering to a toenail or a fingernail of a patient in need of such prophylaxis an amount of a composition comprising
at least one topical antimycotic,
at least one systemic antimycotic, and
at least one physiologically acceptable lacquer base,
wherein the amount is effective for the prophylaxis.

24. A method of treating an infection of a toenail or a fingernail, wherein the method comprises topically administering to a toenail or a fingernail of a patient in need of such treating an amount of a composition comprising
at least one topical antimycotic,
at least one systemic antimycotic, and
at least one physiologically acceptable lacquer base,
wherein the amount is effective for the treating.

25. A method of employing the composition as claimed in claim 1, wherein the method comprises incorporating the composition in a cosmetic composition.
